(19) United States
(12) Patent Application Publication
(10) Pub. No.: US 2025/0151722 A1
(43) Pub. Date: May 15, 2025

(54) COMPOSITION FOR INHIBITING AFLATOXIN PRODUCTION

(71) Applicant: National Agriculture and Foof Research Organization, Tsukuba-shi, Ibaraki (JP)

(72) Inventors: Tomohiro FURUKAWA, Shinagawa-ku, Tokyo (JP); Masayo KUSHIRO, Tsukuba-shi, Ibaraki (JP)

(73) Assignee: National Agriculture and Foof Research Organization, Tsukuba-shi, Ibaraki (JP)

(21) Appl. No.: 18/833,504

(22) PCT Filed: Jan. 18, 2023
(86) PCT No.: PCT/JP2023/001262

§ 371 Date: Jul. 26, 2024

(30) Foreign Application Priority Data
Feb. 1, 2022 (JP) 2022-014052

(51) Int. Cl. A01N 31/02; A01P 3/00

(52) U.S. Cl. CPC A01N 31/02; A01P 3/00

(57) ABSTRACT

The objective of this invention is to provide a novel active ingredient useful for inhibiting aflatoxin production. With this invention, production of aflatoxin by an aflatoxin-producing fungus can be inhibited by means of a primary alcohol represented by formula (1): CH₃—(CH₂)ₙ—OH (1) (where n is 0 or 2-4).

### TECHNICAL FIELD

The present invention relates to a composition for inhibiting aflatoxin production and a method for inhibiting aflatoxin production, and particularly to those in which a specific primary alcohol is an active ingredient.

### BACKGROUND ART

Aflatoxins are mycotoxins with strong toxicity, having carcinogenic properties. The aflatoxin contamination of agricultural products including grains is a major problem worldwide, and causes an enormous economic loss due to the disposal of contaminated food and feed. Since the aflatoxin contamination originates from aflatoxin-producing fungi that are resident in field soil and the like, there is a need to develop technology to control these fungi.

Patent Literature 1 states that dioctatin is usable to inhibit aflatoxin production and control aflatoxin contamination. Non Patent Literature 1 also states that another compound has an antifungal action on aflatoxin-producing fungi and an aflatoxin production inhibitory action. Non Patent Literature 2 suggests that ethanol inhibits aflatoxin B₁ biosynthesis by up-regulating an oxidative stress response in Aspergillus flavus.

### CITATION LIST

### Patent Literature

Patent Literature 1: Japanese Patent Application Publication No. 2019-94261

### Non Patent Literatures

Non Patent Literature 1: JSM Mycotoxins (2019), 69 (2), 81-83
Non Patent Literature 2: Frontiers in Microbiology (2020), 10, Article 2946, doi: 10.3389/fmicb.2019.02946

### SUMMARY OF INVENTION

### Problems to be Solved by the Invention

Various types of fungicides are used as post-harvest pesticides, but their persistence and toxicity are considered as problems, so that there is a demand for safer materials. Therefore, the present invention has an object to provide a novel active ingredient useful for inhibiting aflatoxin production.

### Means for Solving the Problems

As a result of earnest studies to achieve the above object, the present inventors have found that a primary alcohol other than ethanol exerts an aflatoxin production inhibitory action, and thereby completed the present invention. Specifically, the present invention provides a composition for inhibiting aflatoxin production and a method for inhibiting aflatoxin production to be described below.

[1] A composition for inhibiting aflatoxin production by an aflatoxin-producing fungus, comprising a primary alcohol represented by

formula (1): CH₃—(CH₂)ₙ—OH (1),
where n is 0 or 2 to 4.

[2] The composition according to [1], wherein the primary alcohol represented by the formula (1) is at a concentration of 0.1 to 5 v/v %.

[3] The composition according to [1] or [2], wherein the primary alcohol represented by the formula (1) is at least one selected from the group consisting of 1-propanol, methanol, and 1-butanol.

[4] The composition according to any one of [1] to [3], wherein the aflatoxin-producing fungus is a fungus of the genus Aspergillus.

[5] A method for inhibiting aflatoxin production, comprising the step of applying, to an aflatoxin-producing fungus, a composition containing a primary alcohol represented by

formula (1): CH₃—(CH₂)ₙ—OH (1),
where n is 0 or 2 to 5.

[6] The method according to [5], wherein the primary alcohol represented by the formula (1) is at a concentration of 0.1 to 5 v/v %.

[7] The method according to [5] or [6], wherein the primary alcohol represented by the formula (1) is at least one selected from the group consisting of 1-propanol, methanol, and 1-butanol.

[8] The method according to any one of [5] to [7], wherein the aflatoxin-producing fungus is a fungus of the genus Aspergillus.

### Advantageous Effects of Invention

According to the present invention, it is possible to inhibit aflatoxin production by an aflatoxin-producing fungus by using a primary alcohol represented by the above formula (1). Therefore, it is possible to provide safer materials that are less likely to cause problems of persistence and toxicity.

### DESCRIPTION OF EMBODIMENTS

Hereinafter, the present invention will be described further in details.

A composition of the present invention is for use to inhibit aflatoxin production by an aflatoxin-producing fungus, and comprises, as an active ingredient, a primary alcohol represented by

formula (1): CH₃—(CH₂)ₙ—OH (1),
where n is 0 or 2 to 4. The primary alcohol represented by the formula (1) is specifically methanol, 1-propanol, 1-butanol, and/or 1-pentanol, or preferably may be at least one selected from the group consisting of 1-propanol, methanol, and 1-butanol.

The concentration of the primary alcohol represented by the formula (1) is not particularly limited as long as the composition can inhibit the aflatoxin production by an aflatoxin-producing fungus. For example, the concentration of the primary alcohol relative to the composition of the present invention may be about 0.1 v/v % or higher, about 0.2 v/v % or higher, or about 0.5 v/v % or higher, and may be about 5 v/v % or lower, about 1.5 v/v % or lower, or about 0.7 v/v % or lower. In other words, the primary alcohol represented by the formula (1) can produce the effect even if the concentration is low. In a preferred embodiment, when the primary alcohol represented by the formula (1) is methanol, the concentration may be about 0.1 to about 5 v/v % or about 0.8 to about 5 v/v %. When the primary alcohol represented by the formula (1) is 1-propanol, the concentration may be about 0.1 to about 1.5% or about 0.5 to about 1.5 v/v %. When the primary alcohol represented by the formula (1) is 1-butanol or 1-pentanol, the concentration may be about 0.1 to about 0.7% or about 0.5 to about 0.7 v/v %.

In the present specification, “aflatoxins” are a type of mycotoxins, whose known types are B₁, B₂, G₁, G₂, and so on. These aflatoxins are known to be biosynthesized in a common pathway. It is also known that fungi of the genus Aspergillus include fungi that produce aflatoxins, i.e., “aflatoxin-producing fungi”. Examples of the aflatoxin-producing fungus of the genus Aspergillus include A. flavus, A. parasiticus, A. nomius, A. pseudotamarii, A. bombycis, A. parvisclerotigenus, and so on.

In general, use of a chemical substance that has an effect of inhibiting the growth of an aflatoxin-producing fungus has a concern that when a fungus resistant to the chemical substance emerges, the resistant fungus will dominantly grow and spread. However, the primary alcohol represented by the formula (1) has a characteristic of exerting an aflatoxin production inhibitory activity even in a low concentration range where the effect of inhibiting the growth of the aflatoxin-producing fungi is not exerted. For this reason, according to the present invention, it is expected that the problem of the spread of the resistant fungus can be avoided while the aflatoxin contamination of a target is controlled. On the other hand, ethanol, 2-propanol, and 2-methyl-2-propanol, which are not covered by the above formula (1), do not have such effect and rather increase the amount of aflatoxins produced in the low concentration range where the effect of inhibiting the growth of the aflatoxin-producing fungus is not exerted. Without wishing to be bound to any particular theory, it is considered that the primary alcohol represented by the formula (1) can be suitably used because it is not metabolized by the aflatoxin-producing fungus.

The composition of the present invention may further contain an optional solvent and/or additive usually used in this technical field, as long as they do not hinder the object of the present invention, and may further contain any other ingredient which is effective for inhibiting aflatoxin production or for exerting an antifungal activity against an aflatoxin-producing fungus. As the solvent, any solvent commonly used in this technical field may be employed without particular limitation, and, for example, water, a buffer solution, a mixture thereof, or the like may be used.

The composition of the present invention can be used by being applied to a place where an aflatoxin-producing fungus is growing or a place where an aflatoxin-producing fungus is suspected to be growing. Examples of a specific application target include, but not limited to, seeds, plants, harvested agricultural products, fields, and the like.

In another embodiment, the present invention relates to a method for inhibiting aflatoxin production, the method comprising the step of applying, to an aflatoxin-producing fungus, a composition comprising a primary alcohol represented by

formula (1): CH₃—(CH₂)ₙ—OH (1),
where n is 0 or 2 to 5. The composition is as described above regarding the composition of the present invention, and can be also used in the method of the invention in the aforementioned composition form. In an embodiment, the application step may comprise a step of applying the composition to a place where an aflatoxin-producing fungus is growing and/or a place where an aflatoxin-producing fungus is suspected to be growing.

Hereinafter, the present invention will be described specifically by using Examples, but the scope of the present invention should not be limited to these Examples.

### EXAMPLES

### Test Example 1

A. flavus IFM47798 strain was inoculated onto a potato dextrose agar and cultured in the dark at 28° C. for 1 week. Conidia formed on the agar were scraped off with a platinum loop, suspended in a 30% glycerol aqueous solution, and stored at −80° C.

The above conidial suspension was added to and mixed in a potato dextrose liquid medium so that the conidial concentration was 2.5×10⁴ conidia/mL, and added to a 12-well plate at 2 mL per well. Then, alcohols were added, followed by static cultivation in the dark for 48 hours at 28° C. After the cultivation, mycelia were separated by centrifugation, and the supernatant was collected in a tube.

To 0.5 mL of the collected supernatant, 0.5 mL of chloroform was added, followed by mixing with a tube mixer and then centrifuged. The lower chloroform layer was collected in another tube, and evaporated by air drying in a fume chamber. The residue was dissolved in 1 mL of a 90% acetonitrile aqueous solution. Then, aflatoxins B₁, B₂, G₁, and G₂ in the solution were each quantified by a liquid chromatograph-mass spectrometer (LC-MS) and a total value thereof was determined as a total amount of the aflatoxins produced (total AF). Then, a relative value was calculated with a total AF of an untreated control group, to which no alcohol was added, set as 100. The results are shown in Table 1 below.

The collected mycelia in the tube, whose weight had been measured in advance, were frozen in a −80° C. freezer, and lyophilized. The total weight was measured and the tube weight was subtracted to determine the dry fungal weight. Then, a relative value was calculated with the dry fungal weight of the untreated group, to which no alcohol was added, set as 100. Table 1 shows the results. [Table 1]

TABLE 1
Inhibitory Activity of Alcohols on Aflatoxin
Production and Fungal Growth

| | Total AF Relative | Fungal Weight Relative |
|---|---|---|
| Alcohol (v/v %) | Value (%) | Value (%) |
| Methanol | | |
| Untreated (Control) | 100.0 ± 18.1 | 100.0 ± 5.5 |
| 0.3 | 70.6 ± 12.1** | 103.7 ± 3.5 |
| 0.6 | 63.4 ± 12.7*** | 110.1 ± 6.7 |
| 1 | 0.0 ± 0.0**** | 128.4 ± 3.0 |
| 2 | 0.0 ± 0.0**** | 145.9 ± 11.4 |
| 4 | 0.0 ± 0.0 **** | 103.7 ± 3.5 |
| Ethanol | | |
| Untreated (Control) | 100.0 ± 32.7 | 100.0 ± 7.4 |
| 0.3 | 197.1 ± 25.9**** | 111.7 ± 8.1 |
| 0.6 | 244.5 ± 13.2**** | 108.5 ± 10.7 |
| 1 | 222.4 ± 20.9**** | 121.3 ± 5.5 |
| 2 | 24.3 ± 10.5*** | 96.8 ± 8.8 |
| 4 | 0.0 ± 0.0**** | 50.0 ± 5.4 |
| 1-Propanol | | |
| Untreated (Control) | 100.0 ± 6.6 | 100.0 ± 8.4 |
| 0.3 | 10.5 ± 4.0**** | 102.9 ± 12.0 |
| 0.6 | 0.0 ± 0.0**** | 118.1 ± 5.4 |
| 2-Propanol | | |
| Untreated (Control) | 100.0 ± 4.5 | 100.0 ± 5.6 |
| 0.3 | 373.7 ± 21.6**** | 126.2 ± 6.4 |
| 0.6 | 382.4 ± 12.2**** | 128.0 ± 11.6 |
| 1 | 40.4 ± 12.4**** | 103.7 ± 7.7 |
| 2 | 0.0 ± 0.0**** | 94.4 ± 8.3 |
| 4 | 0.0 ± 0.0**** | 0.0 ± 0.0 |
| 2-Butanol | | |
| Untreated (Control) | 100.0 ± 16.0 | 100.0 ± 4.2 |
| 0.3 | 29.9 ± 10.8**** | 66.1 ± 5.4 |
| 0.6 | 0.0 ± 0.0**** | 70.2 ± 4.2 |
| 2-Methyl-2-Propanol | | |
| Untreated (Control) | 100.0 ± 15.7 | 100.0 ± 3.1 |
| 0.3 | 178.7 ± 21.0**** | 104.8 ± 7.9 |
| 0.6 | 156.7 ± 2.7**** | 101.0 ± 4.8 |
| 1 | 0.0 ± 0.0**** | 51.9 ± 15.5 |
| 2 | 0.0 ± 0.0**** | 0.0 ± 0.0 |
| 4 | 0.0 ± 0.0**** | 0.0 ± 0.0 |

For each alcohol, a measurement was performed in n = 4, and the relative value with the mean value of the untreated control group set as 100 is shown. The numerals after ± indicate a sample standard deviation. A two-sided test of a difference from the untreated control group was performed according to Dunnett's test.
**p < 0.01,
***p < 0.001,
****p < 0.0001

When ethanol, which is well known to have an antifungal activity, was applied at a concentration of 4 v/v % or higher, the growth of A. flavus was inhibited and the aflatoxin production was accordingly inhibited. However, when ethanol was applied at a lower concentration, the growth of A. flavus was not inhibited and the amount of aflatoxins produced by A. flavus conversely increased. The same tendencies as in the case of ethanol were observed in the cases of 2-propanol and 2-methyl-2-propanol. In contrast, when the primary alcohols other than ethanol were used at the low concentrations, the aflatoxin production by A. flavus was inhibited in a concentration-dependent manner. Interestingly, even in a low concentration range where A. flavus may grow, use of the primary alcohols other than ethanol led to the inhibition of the aflatoxin production by A. flavus.

### Test Example 2

The total AF and the dry fungal weight were measured and the relative values with respect to the untreated control group were calculated in the same manner as in Test Example 1 except that A. flavus MAFF111229 strain or A. parasiticus NRRL2999 strain was used instead of A. flavus IFM47798 strain and that methanol, 1-propanol, or 1-butanol was used as the alcohol. The results are shown in Tables 2 and 3.

TABLE 2
Activities on A. flavus MAFF111229 strain

| | Total AF Relative | Fungal Weight |
|---|---|---|
| Alcohol (v/v %) | Value (%) | Relative Value (%) |
| Methanol | | |
| Untreated (Control) | 100.0 ± 18.9 | 100.0 ± 16.4 |
| 0.3 | 108.5 ± 5.8 | 98.0 ± 4.6 |
| 0.6 | 102.3 ± 5.0 | 101.4 ± 3.5 |
| 1 | 13.8 ± 0.8**** | 123.6 ± 6.8 |
| 2 | 0.0 ± 0.0**** | 123.6 ± 10.4 |
| 4 | 0.0 ± 0.0**** | 112.8 ± 10.4 |
| 1-Propanol | | |
| Untreated (Control) | 100.0 ± 7.2 | 100.0 ± 3.9 |
| 0.3 | 68.7 ± 2.4**** | 104.5 ± 6.5 |
| 0.6 | 62.0 ± 4.9**** | 103.9 ± 8.8 |
| 2-Butanol | | |
| Untreated (Control) | 100.0 ± 3.3 | 100.0 ± 6.5 |
| 0.3 | 76.2 ± 7.9**** | 56.2 ± 7.3 |
| 0.6 | 24.7 ± 3.6**** | 48.9 ± 8.7 |

For each alcohol, a measurement was performed in n = 4, and the relative value with the mean value of the untreated control group set as 100 is shown. The numerals after ± indicate a sample standard deviation. A two-sided test of a difference from the untreated control group was performed according to Dunnett's test.
*p < 0.05,
**p < 0.01,
***p < 0.001,
****p < 0.0001

TABLE 3
Activities on A. parasiticus NRRL2999 strain

| | Total AF Relative | Fungal Weight Relative |
|---|---|---|
| Alcohol (v/v %) | Value (%) | Value (%) |
| Methanol | | |
| Untreated (Control) | 100.0 ± 1.7 | 100.0 ± 2.2 |
| 0.3 | 86.3 ± 3.6**** | 102.1 ± 1.6 |
| 0.6 | 72.3 ± 1.9**** | 102.9 ± 3.7 |
| 1 | 31.1 ± 1.4**** | 93.3 ± 11.2 |
| 2 | 10.3 ± 1.3**** | 92.0 ± 6.9 |
| 4 | 3.4 ± 0.8**** | 100.0 ± 8.5 |
| 1-Propanol | | |
| Untreated (Control) | 100.0 ± 4.6 | 100.0 ± 2.4 |
| 0.3 | 58.7 ± 9.5**** | 87.4 ± 1.8 |
| 0.6 | 47.5 ± 6.5**** | 86.4 ± 2.8 |
| 2-Butanol | | |
| Untreated (Control) | 100.0 ± 3.7 | 100.0 ± 4.7 |
| 0.3 | 54.6 ± 4.6**** | 43.9 ± 2.7 |
| 0.6 | 0.0 ± 0.0**** | 9.0 ± 4.0 |

For each alcohol, a measurement was performed in n = 4, and the relative value with the mean value of the untreated control group set as 100 is shown. The numerals after ± indicate a sample standard deviation. A two-sided test of a difference from the untreated control group was performed according to Dunnett's test.
****p < 0.0001

Even if the type of fungi tested was changed to a different strain or species, the characteristics of the primary alcohols other than ethanol was observed in which they exerted the aflatoxin production inhibitory activity in the lower concentration where the growth of aflatoxin-producing fungi was not completely inhibited. Among these, 1-propanol is particularly very useful in preventing aflatoxin contamination of agricultural products, as 1-propanol is effective at low concentration and is also used as a food additive.

### Test Example 3

Ten g of corn kernels were placed in Petri dish, and was inoculated with 0.1 mL/Petri dish of a conidial suspension of A. flavus MAFF111229 strain (a conidial concentration of 1×10⁶ spores/mL). Methanol, 1-propanol or 1-butanol was sprayed onto the kernel surfaces at 0.2 mL/petri dish (20 μL per 1 g of kernels) by using a spray vial. A group that was not sprayed with any alcohol was prepared as a positive control, and a group that was not inoculated with the fungi and not sprayed with any alcohol was prepared as a negative control. The kernels from each test group were placed on an agar plate, the plate was wrapped with a parafilm, and allowed to stand at 28° C. for one week.

The kernels of each test group were collected and ground by a blender, 40 mL of 90% acetonitrile was added, mixed for 30 minutes in a shaker, and then centrifuged to collect the supernatant. 5 mL of the supernatant was supplied to a solid-phase extraction column Autoprep (R) MF-A1000 (manufactured by Showa Denko K. K.) and the first 2 mL fluid passed was collected. After drying by nitrogen blowing, the residue was dissolved in 0.5 mL of 90% acetonitrile. Then, aflatoxins B₁, B₂, G₁, and G₂ in this solution were each quantified by the LC-MS and a total value thereof was determined as a total amount of aflatoxins produced (total AF). Then, a relative value was calculated with a total AF of the positive control group set as 100. The results are shown in Table 4.

TABLE 4
Aflatoxin Inhibitory Activity on Corn Kernels

| Alcohol | Total AF Relative Value (%) |
|---|---|
| Positive Control Group | 100.0 ± 13.4 |
| Methanol | 1.0 ± 1.3**** |
| 1-Propanol | 0.0 ± 0.0**** |
| 2-Butanol | 0.0 ± 0.0**** |
| Negative Control Group | 0.0 ± 0.0**** |

For each alcohol, a measurement was performed in n = 3, and the relative value with the mean value of the positive control group set as 100 is shown. The numerals after ± indicate a sample standard deviation. A two-sided test of a difference from the untreated control group was performed according to Dunnett's test.
****p < 0.0001

It was confirmed that the alcohols that exerted the aflatoxin production inhibitory activity in Text Examples 1 and 2 were able to inhibit the aflatoxin production also on the corn kernels.

From the above, it was understood that the primary alcohol represented by the formula (1) is able to inhibit aflatoxin production by an aflatoxin-producing fungus. Therefore, it is possible to provide safer materials that are less likely to cause problems of persistence and toxicity.

1. A composition for inhibiting aflatoxin production by an aflatoxin-producing fungus, comprising a primary alcohol represented by formula (1): CH₃—(CH₂)ₙ—OH (1), where n is 0 or 2 to 4.

2. The composition according to claim 1, wherein the primary alcohol represented by the formula (1) is at a concentration of 0.1 to 5 v/v %.

3. The composition according to claim 1, wherein the primary alcohol represented by the formula (1) is at least one selected from the group consisting of 1-propanol, methanol, and 1-butanol.

4. The composition according to claim 1, wherein the aflatoxin-producing fungus is a fungus of the genus Aspergillus.

5. A method for inhibiting aflatoxin production, comprising the step of applying, to an aflatoxin-producing fungus, a composition comprising a primary alcohol represented by formula (1): CH₃—(CH₂)ₙ—OH (1), where n is 0 or 2 to 4.

6. The method according to claim 5, wherein the primary alcohol represented by the formula (1) is at a concentration of 0.1 to 5 v/v %.

7. The method according to claim 5, wherein the primary alcohol represented by the formula (1) is at least one selected from the group consisting of 1-propanol, methanol, and 1-butanol.

8. The method according to claim 5, wherein the aflatoxin-producing fungus is a fungus of the genus Aspergillus.
